(19) United States
(12) Patent Application Publication
(10) Pub. No.: US 2010/0160906 A1
(43) Pub. Date: Jun. 24, 2010

(54) EXPANDABLE ENERGY DELIVERY DEVICES HAVING FLEXIBLE CONDUCTIVE ELEMENTS AND ASSOCIATED SYSTEMS AND METHODS

(75) Inventor: Jerry Jarrard, Sunnyvale, CA (US)

Correspondence Address: PERKINS COIE LLP;PATENT SEA, P.O. BOX 1247, SEATTLE, WA, 98111, US

(73) Assignee: Asthmatx, Inc., Sunnyvale, CA (US)

(21) Appl. No.: 12/640,644

(22) Filed: Dec. 17, 2009

Related U.S. Application Data
(60) Provisional application No. 61/140,547, filed on Dec. 23, 2008.

(51) Int. Cl. A61B 18/14; A61B 18/18; B05D 5/06

(52) U.S. Cl. 606/33; 606/41; 427/2.1

(57) ABSTRACT

Devices, systems, and methods are disclosed for providing energy to tissue in an internal passageway. An energy delivery device for treating an airway in a patient in accordance with one embodiment of the disclosure can include an elongated support configured to be passed through a lumen of a scope and a compliant inflatable member attached to the elongated support. The inflatable member is configured to move between a collapsed configuration and an expanded configuration. The energy delivery device can also include a flexible conductive element carried by the inflatable member. The flexible conductive element extends along a path and is configured to expand and contract longitudinally relative to the path as the inflatable member moves between the expanded and collapsed configurations.

### CROSS-REFERENCE TO RELATED APPLICATION

The present application claims priority under 35 U.S.C. §119(e) to U.S. Provisional Application Ser. No. 61/140,547, filed Dec. 23, 2008, the disclosure of which is incorporated herein by reference in its entirety.

### TECHNICAL FIELD

The present application relates generally to medical treatment devices, such as devices that treat lung diseases by applying energy to airways to reduce the resistance to airflow in the airways.

### BACKGROUND

Asthma is a disease that makes it difficult to breathe and in many cases can be debilitating. Asthma is generally manifested by (a) bronchoconstriction, (b) excessive mucus production, and/or (c) inflammation and swelling of airways that cause widespread but variable airflow obstructions. Asthma can be a chronic disorder often characterized by persistent airway inflammation, but asthma can be further characterized by acute episodes of additional airway narrowing via contraction of hyper-responsive airway smooth muscle tissue.

Conventional pharmacological approaches for managing asthma include (a) administering anti-inflammatories and long-acting bronchodilators for long-term control, and/or (b) administering short-acting bronchodilators for management of acute episodes. Both of these pharmacological approaches generally require repeated use of the prescribed drugs at regular intervals throughout long periods of time. However, high doses of corticosteroid anti-inflammatory drugs can have serious side effects that require careful management, and some patients are resistant to steroid treatment even at high doses. As such, effective patient compliance with pharmacologic management and avoiding stimuli that trigger asthma are common barriers to successfully managing asthma.

Asthmatx, Inc. has developed new asthma treatments that involve applying energy to alter properties of the smooth muscle tissue or other tissue (e.g., nerves, mucus glands, epithelium, blood vessels, etc.) of airways in a lung of a patient. Several embodiments of methods and apparatus related to such treatments are disclosed in commonly assigned U.S. Pat. Nos. 6,411,852, 6,488,673, 6,634,363, 7,027,869, 7,104,987 and 7,425,212; and U.S. Patent Application Publication Nos. US 2005/0010270 and US 2006/0247746, all of which are incorporated by reference herein in their entirety.

Many embodiments of the foregoing asthma treatments that apply energy to tissue of the airways use catheters that can be passed (e.g., navigated) through the tortuous passageways defined by the lung airways. FIG. 1, for example, illustrates a bronchial tree 90 of a lung in which the various bronchioles 92 decrease in size as they extend from branches 96 that in turn extend from the right and left bronchi 94. Accordingly, the treatment devices should be configured to treat airways of varying sizes as well as to function properly when repeatedly deployed after navigating through the tortuous anatomy.

One challenge of delivering energy to the airways in the lung is that it may take three sessions of 30-60 minutes each to treat a substantial portion of the lungs of a patient (e.g., upper and lower lobes). The three treatment sessions are usually performed on separate days, so it is also desirable to reduce the time necessary for such treatments. One factor affecting the treatment time is the length or width of the electrodes that contact the airway tissue. Another factor affecting treatment time is the loss of energy to surrounding tissue when monopolar configurations are employed.

Typical monopolar energy delivery devices require about 10 second activation time periods at approximately 65° C. to sufficiently treat target tissue and have small, short electrodes that limit the size of the contact area to reduce or mitigate non-uniformities of the current density in the tissue at the electrode. More specifically, the difference between the current density in the tissue at an edge of an electrode and the center of the electrode increases with increasing electrode dimensions (e.g., electrode length) due in part to tissue resistivity characteristics. The current density in the tissue proximate to the edges of a large, long electrode is accordingly significantly higher than the current density in the tissue at the center of the long electrode. This is known as the “edge effect,” and it is generally undesirable because the higher current densities at the edges or ends of the long electrode (or several short electrodes axially spaced and effectively acting as one long conductive area) may ablate and/or otherwise affect the airway tissue in an undesirable manner.

Current monopolar electrode configurations are accordingly limited to relatively small electrodes (e.g., 3-5 mm long) that exhibit acceptable edge effect non-uniformities. Small electrodes, however, treat corresponding small regions of tissue. Accordingly, small electrodes are advanced axially along airways in a large number of small increments to treat long segments of airways throughout the lung of the patient. As a result, small electrodes require longer and/or more treatment sessions to treat a patient and may result in over/under treatment of long segments due to repeated re-positioning.

### BRIEF DESCRIPTION OF THE DRAWINGS

FIG. 1 illustrates airways within a human lung.

FIG. 2 is a schematic view illustrating a system with a power/control unit, an inflation source, and an energy delivery device for delivering energy to tissue in accordance with several embodiments of the disclosure.

FIGS. 3A and 3B are partially schematic, side cross-sectional views illustrating an energy delivery unit having flexible conductive elements configured in accordance with an embodiment of the disclosure.

FIGS. 4A-5B are enlarged, schematic illustrations of conductive inks used to form flexible conductive elements in accordance with several embodiments of the disclosure.

FIG. 6 is a partially schematic, side cross-sectional view illustrating an energy delivery unit having flexible conductive elements configured in accordance with another embodiment of the disclosure.

FIG. 7 is a partially schematic, side cross-sectional view illustrating an energy delivery unit having flexible conductive elements configured in accordance with still another embodiment of the disclosure.

FIG. 8 is a partially schematic, side cross-sectional view illustrating an energy delivery unit having flexible conductive elements configured in accordance with yet another embodiment of the disclosure.

FIGS. 9A and 9B are partially schematic, side cross-sectional view illustrating an energy delivery unit having inelastic conductive elements configured in accordance with an embodiment of the disclosure.

### DETAILED DESCRIPTION

### A. Overview

Aspects of the present disclosure are directed to devices, systems, and methods for controlling the delivery of energy to tissue within a body cavity. Several examples of such systems have an energy delivery device configured to control the current density in tissue along an extended length. In particular, bipolar energy delivery devices do not exhibit edge effects, and as such efficiency does not decline with electrode length. Further, bipolar configurations provide faster, more efficient treatment because energy is concentrated in the target tissue resulting in more effective energy delivery. Thus, several embodiments of systems in accordance with this disclosure have relatively larger or longer conductive areas that can treat large cross-sectional areas of tissue within shorter activation time periods to reduce the number of treatment sites and cycles compared to shorter or otherwise smaller monopolar electrodes. Accordingly, shorter and/or fewer treatment sessions are needed to treat a patient and improved treatment consistency is achieved.

Specific examples of the system include an energy delivery device having an elongated member and an energy delivery unit at a distal portion of the elongated member. The energy delivery unit can include an inflatable or expandable, compliant member (e.g., a balloon) and one or more flexible, elongatable conductive elements or traces on an outer surface of the inflatable member. The flexible conductive elements can be composed of an electrically conductive, elastic material that stretches with the inflatable member without significant loss of conductivity. In this way, the flexible conductive element(s) effectively function as electrodes, while being simpler and less expensive to manufacture. The flexible conductive elements can have varying sizes, shapes, physical properties, numbers, thickness, spacing, distribution and/or densities. For example, the flexible conductive elements may be composed of conductive inks, polymers, or other suitable materials. The conductive elements can be disposed on the corresponding inflatable member using a variety of different processes. The flexible conductive elements may also be composed of an inelastic material (e.g., wire) configured to longitudinally expand and contract with the inflatable member.

Specific details of several embodiments of treatment devices, systems, and methods for delivering energy to passageways in a patient are described below with respect to delivering radio frequency energy to airways in a lung of a patient to treat asthma. Other embodiments of the technology, however, can deliver other energy modalities to lung airways or other tissues (e.g., body cavities, nerves, glands, skin, etc.) or passageways (e.g., blood vessel) for treating other indications. For example, the system can be configured to deliver thermal (resistive and/or infrared), laser, microwave, ultrasonic (e.g., HIFU), cryo-ablation, mechanical or other types of energy modalities to tissue. Moreover, several other embodiments of the invention can have different configurations, components, or procedures than those described in this section. As such, several of the details set forth below are provided to describe the following examples in a manner sufficient to enable a person skilled in the relevant art to practice, make and use the described examples without undue experimentation. Several of the details and advantages described below, however, may not be necessary to practice certain embodiments of the technology. Additionally, the technology may include other embodiments and methods that are within the scope of the claims but are not described in detail. Moreover, the particular features, structures, routines, steps, or characteristics may be combined in any suitable manner in one or more embodiments of the technology.

### B. Embodiments of Treatment Systems

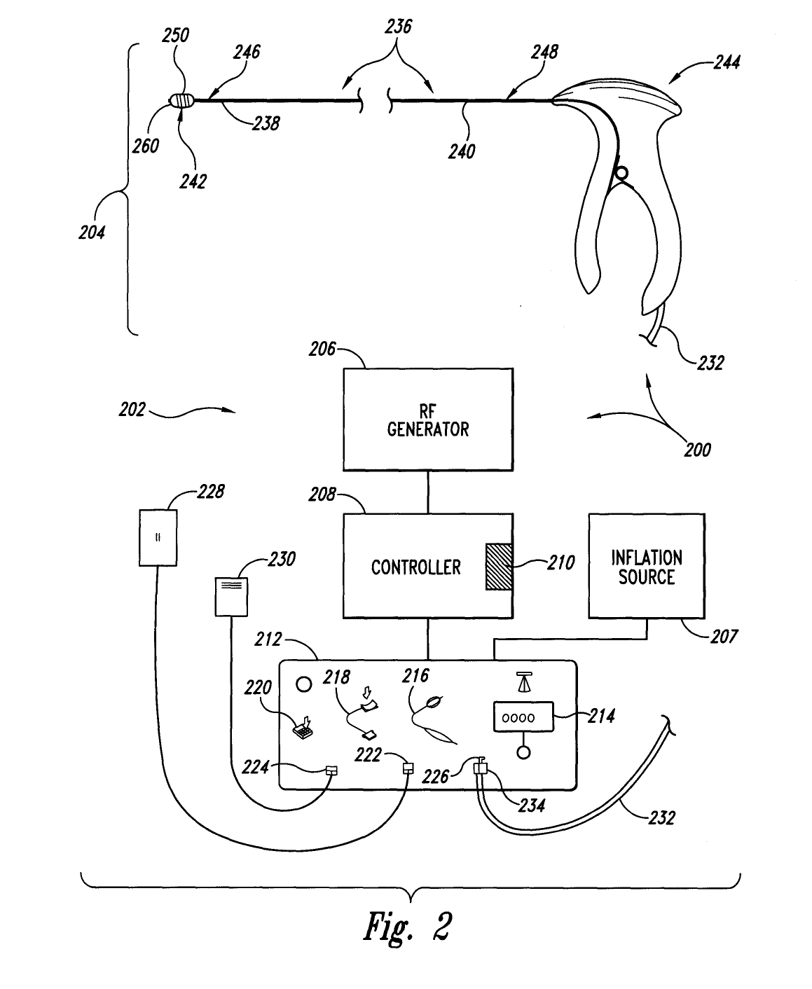

FIG. 2 is a schematic view illustrating a system 200 with a power/control unit 202, an inflation source 207, and an energy delivery device 204 for delivering energy to passageways in a patient in accordance with several embodiments of the disclosure. The power/control unit 202 can include an energy generator 206 (e.g., power supply), a controller 208 having a processor 210, and a user interface 212. The energy generator 206 and the controller 208 can provide radio frequency (RF) energy to the energy delivery device 204, but in other embodiments the energy generator 206 and the controller 208 can provide other energy modalities. The controller 208 can contain safety algorithms and other control algorithms that control (a) the power output to the energy delivery device 204 and (b) indicators 214, 216, 218, and 220 of the user interface 212. The power/control unit 202 can further include one or more connections 222, 224, and 226 for an optional return electrode 228 for monopolar RF configurations, an optional switch 230 (e.g., an actuation pedal) for directing the controller 208 to cause the energy generator 206 to provide energy, and a conductive line 232 and a connector 234 coupled to the energy delivery device 204. The inflation source 207 may also be coupled to the user interface 212 to provide inflation medium (e.g., gas, liquid, etc.) to the inflatable member 260 via line 232. Alternatively, the inflation source 207 (e.g., syringe) may be directly incorporated into the catheter handles 244 as opposed to the user interface 212. It will be appreciated that the depictions herein are for illustrative purposes only and do not necessarily reflect the actual shape, size, or dimensions of the system or device.

The energy delivery device 204 is an example of a treatment device for treating asthma or other indications associated with passageways in a human. The embodiment of the energy delivery device 204 illustrated in FIG. 2 includes an elongated body 236 with a distal portion 238 and a proximal portion 240, an energy delivery unit 242 at the distal portion 238, and a handle 244 at the proximal portion 240. The length of the elongated body 236 should be sufficient to access the target tissue in airways of the lung or other passageways targeted for treatment. For example, the length of the elongated body 236 can be from approximately 0.5-8.0 feet to allow passage through a bronchoscope and reach of targeted airways deep within the lungs. The elongated body 236 can be configured to treat airways as small as 2 mm in diameter, but the elongated body 236 is not limited to treating airways of any particular size such that airways smaller or larger than 2 mm may be treated. Typically, the energy delivery unit 242 expands/contracts to variable sizes to treat airways between 2-10 mm.

Several embodiments of the elongated body 236 are flexible catheters configured to slide through the working lumen of an access device (e.g., bronchoscope; not shown in FIG. 2). The elongated body 236 can also include a plurality of markers 246 at the distal portion 238 to position the energy delivery unit 242 relative to an access device and a proximal marker(s) 248 so as to assist in expedient positioning of the energy delivery unit 242 out of the distal end of the access device. Specific embodiments of elongated bodies 236 with markers 246 suitable for use in the system 200 are described in U.S. Patent Application Publication Nos. US 2007/0106292 and US 2008/0097424, all of which are incorporated herein by reference in their entirety.

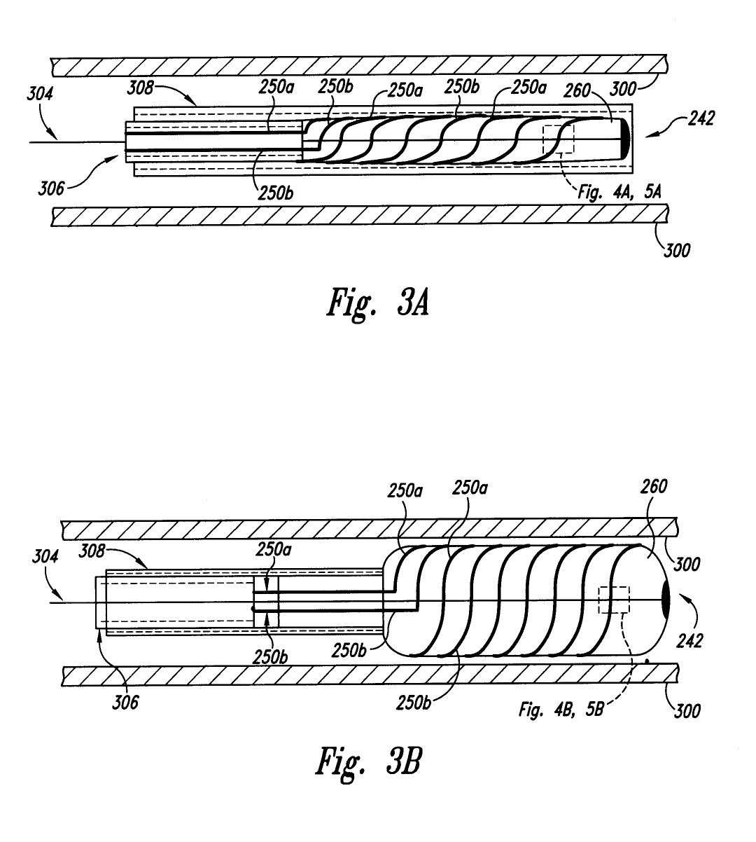

The energy delivery unit 242 can have an inflatable member 260 movable between a retracted and an expanded configuration and one or more flexible conductive elements 250 configured to deliver energy to the tissue of an airway, passageway, or other body cavity in the patient. The inflatable member 260, for example, can be configured to contact the passageway and present the conductive elements 250 to the passageway. For example, the inflatable member 260 may comprise a compliant balloon or another type of member that is inflatable, self-expandable, and/or mechanically actuatable. The inflatable member may expand anywhere from two to three times from its initial diameter so as to treat airways in a range from 2-6 mm and 2-8 mm, respectively. The conductive elements 250 can be flexible or expandable metal bands, metallic arrays, traces, electrode segments, or other electrically conductive mediums or elements that can conduct sufficient electrical current in applications that deliver RF energy or other electrical energy. Further details describing various embodiments of suitable energy delivery units 242 are described below with reference to FIGS. 3A-9B.

The handle 244 is configured so that a single operator can hold an access device (e.g., a bronchoscope) in one hand (e.g., a first hand) and use the other hand (e.g., a second hand) to (a) advance the elongated body 236 through a working lumen of the access device until the energy delivery unit 242 projects beyond the distal end of the access device and is positioned at a desired target site and (b) inflate or otherwise expand the inflatable member 260 to move the conductive elements 250 outwardly until they contact the sidewall of an airway passage while the catheter is held in place relative to the access device with the same second hand. The same operator can also operate the switch 230 of the power/control unit 202 such that the entire procedure can be performed by a single person. The handle 244 is described in greater detail in U.S. patent application Ser. No. 11/777,225, which is incorporated by reference herein in its entirety.

In several embodiments of the system, the controller 208 includes a processor that is generally configured to accept information from the system 200 and components associated with the system 200. The processor can process the information according to various algorithms to produce control signals for controlling the energy generator and/or produce information signals. The information signals produced by the processor may be directed to visual indicators, a digital display, or an audio tone generator of the user interface to inform the user of the system status, component status, procedure status, or any other useful information monitored by the system. The processor of the controller 208 may be a digital IC processor, analog processor, or any other suitable logic or control system that carries out the control algorithms.

Specific embodiments of systems that control power output to the energy delivery device 204 are described in U.S. Pat. No. 7,104,987, U.S. Patent Application Publication No. US 2006/0247746, and U.S. patent application Ser. No. 12/179,301, which are incorporated by reference herein in their entirety. The system 200 may deliver energy to target sites via the energy delivery device 204 in a variety of treatment patterns. Further details with respect to other designs and types of treatment devices, examples of energy, and/or examples of treatment patterns may be found in commonly-assigned U.S. Pat. No. 6,411,852.

### C. Embodiments of Energy Delivery Units Having Flexible Conductive Elements

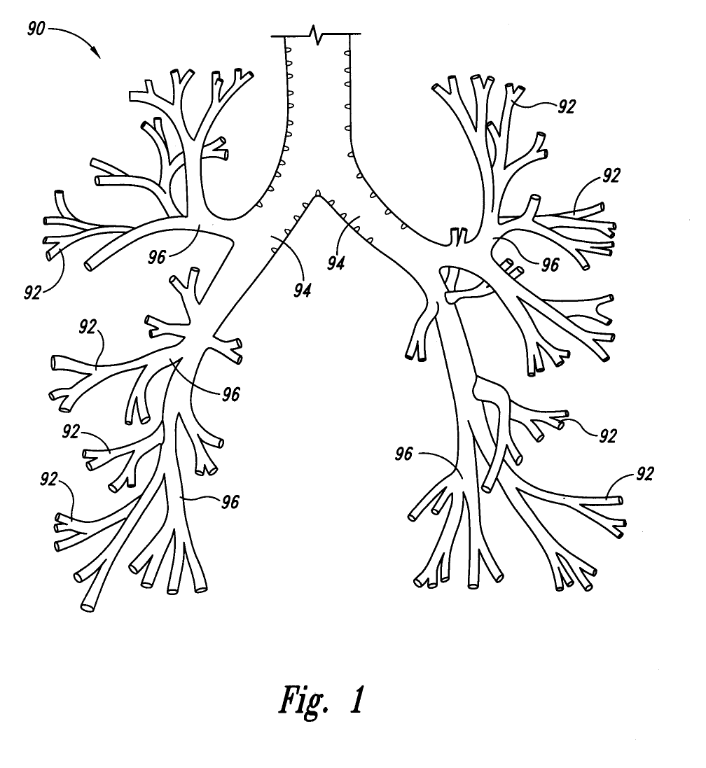

FIGS. 3A and 3B are partially schematic, side cross-sectional views illustrating one example of the energy delivery unit 242 of FIG. 2 after insertion of the unit into a passageway 300 (e.g., an airway as shown in FIG. 1). More specifically, FIG. 3A illustrates the energy delivery unit 242 in a retracted or undeployed configuration, and FIG. 3B illustrates the energy delivery unit 242 in an expanded or deployed configuration. Referring to FIGS. 3A and 3B together, the energy delivery unit 242 includes an inner member 304, an outer member 306, and a sheath 308. The inflatable member 260 is coupled to the outer member 306. As described in greater detail below, the inner member 304 can be a tube, shaft, or helical coil configured to be used as a push rod to help deploy the inflatable member 260 from the retractable sheath 308. It will be appreciated that the energy delivery unit 242 may be free of the inner member 304 and/or sheath 308 components. The outer member 306 may be used to inflate the inflatable member 260 with a gas (e.g., air) or a liquid (e.g., saline). In several embodiments, the outer member 306 is a tube that can also supply cooling medium (e.g., saline, air, etc.) to the inflatable member 260. The fluid can be used to cool the energy delivery unit 242 and adjacent tissue during a treatment session.

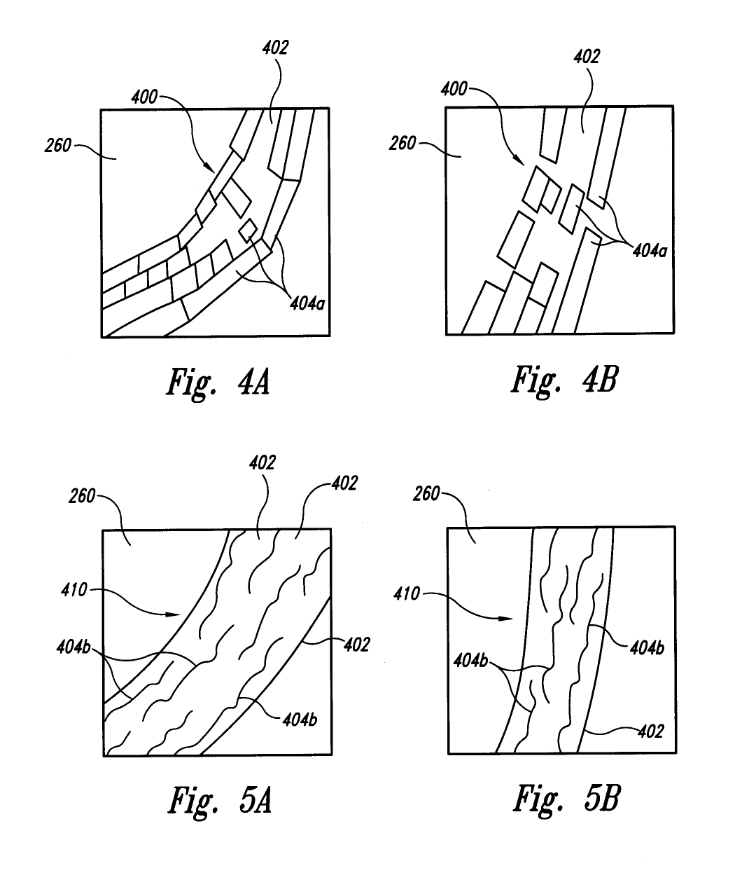

The inflatable member 260 comprises a non-conductive, non-porous, compliant balloon. The inflatable member 260 can be composed of a material such as silicone, a low durometer polymer (e.g., urethane), or another suitable biocompatible material that allows the inflatable member 260 to expand (e.g., approximately 2-3 times) from its original diameter. In the illustrated embodiment, the energy delivery unit 242 includes two flexible, elongatable conductive elements 250a and 250b carried by an outer surface of the inflatable member 260. The conductive elements 250a and 250b of this embodiment are composed of an elastic material that stretches with the inflatable member 260 (such as in the expanded configuration of FIG. 3B) without significant loss of conductivity. For example, the conductive elements 250a and 250b can be composed of conductive inks, as described in further detail below with reference to FIGS. 4A-5B. In the illustrated embodiment, the conductive elements 250a and 250b are applied in a helical formation around the inflatable member 260 so they perform like a series of bipolar rings so as to provide complete circumferential and longitudinal heating of target tissue. In other embodiments, however, the conductive elements may have a different configuration on the inflatable member (e.g., interdigitated, serpentine, accordion, etc.).

In operation, the energy delivery unit 242 is inserted into the passageway 300 in the retracted configuration (as shown in FIG. 3A). In this arrangement, the inflatable member 260 is completely encased in the sheath 308. After moving the energy delivery unit 242 through the passageway 300 to a desired treatment location, the inflatable member 260 can be deployed to the expanded configuration (as shown in FIG. 3B). For example, as described previously, the inner member 304 can be used as a push rod to move the inflatable member 260 out of the sheath while the outer member 306 inflates the inflatable member 260 to an expanded configuration at a desired pressure and/or diameter and into contact with a wall of the passageway 300.

After the inflatable member 260 is expanded, the power control unit 202 (FIG. 2) provides power to the energy delivery unit 242. More specifically, an RF voltage is applied between the conductive elements 250a and 250b in a bipolar manner causing RF current to flow between the conductive elements 250a and 250b and heating of the passageway 300. The inflatable member 260 can subsequently be deflated and/or moved back to the retracted configuration by holding the inner member 304 stationary while proximally pulling the outer member 306 to reduce the diameter of the inflatable member 260 until it fits inside the sheath 308. The energy delivery unit 242 can then be moved to another desired treatment location or removed from the passageway 300.

One feature of the bipolar energy delivery unit 242 described above is that the depth and uniformity of the treatment area can be increased as compared with conventional monopolar energy delivery devices. For example, the bipolar design of the energy delivery unit 242 allows the full circumference of the passageway (e.g., airway) to be treated. Thus, the energy delivery unit 242 may provide a uniform or near uniform current density in tissue around the circumference and/or along a length of a bronchial airway or other passageway. In addition, more energy can be delivered to the passageway during each treatment session because the longitudinal coverage of the conductive elements 250a and 250b along the airway wall can be far greater than the 5 mm maximum length of monopolar electrodes (e.g., 5-10-15 mm) without the effectiveness of the conductive elements being hampered by edge effects. Accordingly, the total treatment time for a patient can be reduced. Moreover, less total energy is required than with conventional monopolar devices because the energy loss to the surrounding tissue is expected to be reduced as energy is concentrated in the treatment area. As such, activation time periods in bipolar mode can be reduced (e.g., 1-4 seconds at approximately 60-75° C.; 2-3 seconds at approximately 65-70° C.).

Another feature of the energy delivery unit 242 is that the spacing between the conductive elements 250a and 250b can be precisely controlled. For example, the depth of heating can be adjusted by varying the conductivity, width, and/or spacing of the conductive elements 250a and 250b on the surface of the inflatable member 260. This feature is expected to provide better control and enhance the ability to produce generally uniform treatment areas about the energy delivery unit 242.

Still another feature of the energy delivery unit 242 described above is that a range of airway sizes can be treated with a single design. For example, because the flexible conductive elements 250a and 250b can stretch or expand with the inflatable member 260, the energy delivery unit 242 can be used in airways having varying geometries. Accordingly, a single design can be used for treating airways having a wide range of sizes and/or shapes.

FIGS. 4A-5B are enlarged, schematic illustrations of the areas 4A-5B shown in FIGS. 3A and 3B. More specifically, FIGS. 4A-5B illustrate conductive inks used to form the flexible conductive elements 250a and 250b in accordance with several embodiments of the disclosure. In FIGS. 4A and 5A, the conductive inks are at an initial or unexpanded state (such as when the energy delivery unit 242 is in the collapsed configuration of FIG. 3A), and in FIGS. 4B and 5B the conductive inks are stretched and fully expanded (such as when the energy delivery unit 242 is in the expanded configuration of FIG. 3B).

Referring first to FIG. 4A, a suitable conductive ink 400 can be composed of a binder or base 402 and a conductive filler 404a dispersed in the binder 402. The binder 402 can be composed of a flexible, compliant polymer (e.g., urethane), silicone, or another suitable biocompatible material configured to stretch with the inflatable member 260 (FIGS. 3A and 3B) during device operation. The conductive filler 404a can be particles that have a variety of different sizes, shapes, distributions, and/or concentrations within the binder 402. For example, the conductive filler 404a of FIG. 4A comprises a large number of small and large conductive flakes. Referring next to FIG. 5A, another suitable conductive ink 410 can include the binder 402 and a conductive filler 404b. In this embodiment, the conductive filler 404b comprises conductive fibers or strands. In still other embodiments, the conductive filler 404a-b can be spheres, rods, cylinders, strips, pellets, or combinations thereof. The individual particles or fibers of the conductive filler 404a-b may further be oriented to slide over one another (e.g., overlapping) to maintain contact as the corresponding conductive ink 400 and 410 stretches and expands during operation so as to ensure conductive continuity and durability. The conductive filler 404 can be composed of silver, gold, copper, carbon, or other suitable biocompatible conductive materials. Suitable conductive inks are commercially available from Creative Materials, Inc. of Tyngsboro, Mass.

The conductive ink 400 and 410 may be applied to the inflatable member 260 using a pen, a silk screen process, a pad print process, a spraying process, an ink jet printing process, or other suitable techniques. In one embodiment, for example, the conductive ink 400 and 410 can be applied to the inflatable member 260 and cured while the inflatable member 260 is in an expanded position. In some situations, however, the conductive ink 400 and 410 may separate, tent, or crack when applied to the inflatable member 260 in a fully expanded position. Accordingly, in other embodiments, the inflatable member 260 can be expanded to an intermediate (or semi-expanded) position for application of the conductive ink 400 and 410. For example, if conductive ink 400 and 410 is being applied to an inflatable member 260 that has a diameter of approximately 2 mm in its retracted configuration (e.g., FIG. 3A) and a diameter of approximately 8 mm in its expanded configuration (e.g., FIG. 3B), the inflatable member 260 can be expanded to a diameter of approximately 3-6 mm for application and optimal adhesion of the conductive ink 400 and 410. The conductive ink 400 and 410 and other suitable conductive inks can be applied using the techniques described above or other suitable techniques.

The conductive filler 404a-b in the conductive inks 400 and 410 changes in resistance when the corresponding inflatable member (e.g., inflatable member 260 of FIGS. 3A and 3B) expands. Thus, the volume and configuration (e.g., size, shape, distribution) of the conductive filler 404a-b in the corresponding inks 400 and 410 can be adjusted so that the resistance and, accordingly, the conductivity of the conductive elements 250a and 250b (FIG. 3B) can be selected to vary within a specified range during stretching and expansion. In one embodiment, for example, the resistance of the conductive elements 250a and 250b is about 1 ohm when the inflatable member 260 is in a retracted configuration (FIG. 3A) and about no more than 10 ohms when the inflatable member 260 is in an expanded configuration (FIG. 3B). Minimization of resistance variation between configurations is important to efficiently and uniformly heat the target tissue. In certain embodiments, the conductive inks 400 and 410 have a conductivity that is approximately at least an order of magnitude greater than the tissue of a portion of the passageway (e.g., the passageway 300 of FIGS. 3A and 3B) that is adjacent to the corresponding energy delivery unit (e.g., the energy delivery unit 242 of FIGS. 3A and 3B).

In still other embodiments, the conductive inks 400 and/or 410 can include different features and/or have different configurations. For example, in one embodiment the individual elements of the conductive particles 404a-b may be aligned or at least approximately aligned to minimize the resistance of the corresponding conductive ink 400 and 410 when it is fully stretched. In another embodiment, the conductive ink 400 and 410 may be overloaded with the conductive particles 404a-b so that the resistance of the conductive ink 400 and 410 is greater when the conductive ink is in its initial, unstretched state than when the conductive ink 400 and 410 stretches as the inflatable member 260 is inflated.

### D. Additional Embodiments of Energy Delivery Units Having Flexible Conductive Elements

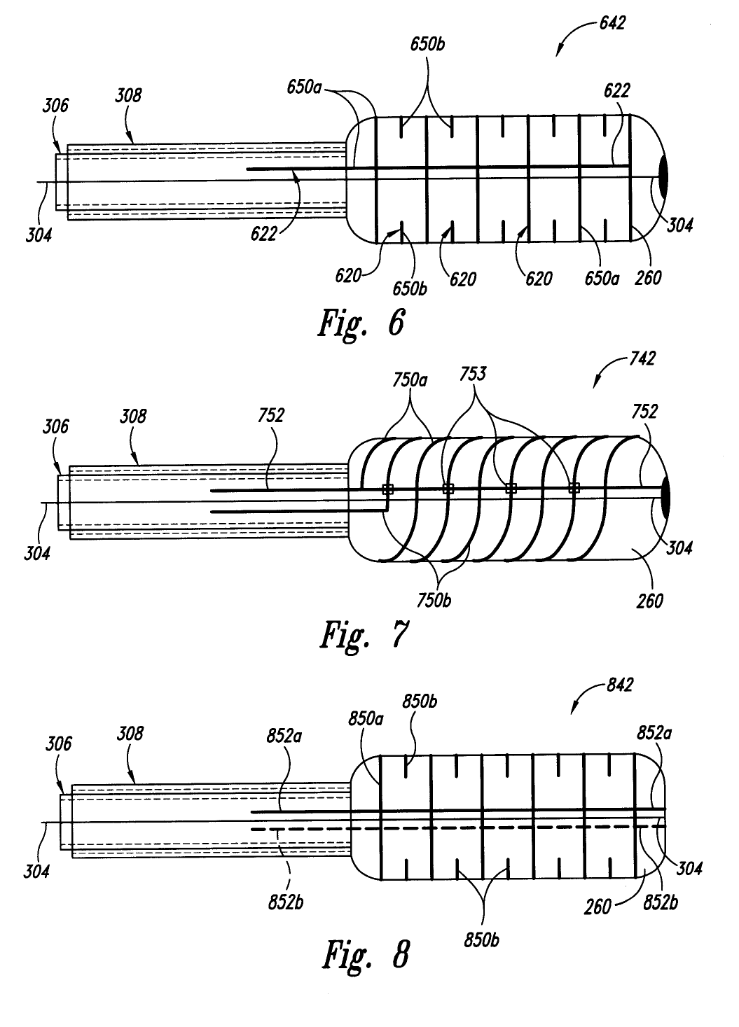

FIGS. 6-9B are partially schematic, side cross-sectional views illustrating energy delivery units having flexible conductive elements configured in accordance with other embodiments of the disclosure. The energy delivery units of FIGS. 6-9B can be used with the system 200 or other suitable systems. In addition, the energy delivery units described below can include many of the same features and advantages of the energy delivery unit 242 described above with reference to FIGS. 2-5B.

FIG. 6, for example, is a side cross-sectional view of an energy delivery unit 642 having flexible conductive elements 650a and 650b configured in accordance with another embodiment of the disclosure. In this embodiment, for example, the conductive elements 650a and 650b are applied to the inflatable member 260 in an interdigitated or interwoven arrangement. The conductive elements 650a and 650b can include, for example, both transverse, radial finger portions 620 and longitudinal finger portions 622. In operation, the conductive elements 650a are biased at one polarity and the conductive elements 650b are biased at an opposite polarity to provide a virtual ring-like bipolar RF field in the airway tissue.

FIG. 7 is a partially schematic, side cross-sectional view of an energy delivery unit 742 configured in accordance with still another embodiment of the disclosure. The energy delivery unit 742 includes flexible conductive elements 750a and 750b arranged generally similar to the conductive elements 250a and 250a of the energy delivery unit 242 of FIGS. 3A and 3B (e.g., a helical arrangement around the inflatable member 260). The energy delivery unit 742 differs from the energy delivery unit 242, however, in that the energy delivery unit 742 further includes a non-elongatable conductive element 752 (e.g., a wire, non-compliant base layer with conductive element) attached to or carried by the inflatable member 260. The non-elongatable conductive element 752 is highly conductive and connected to the distal portion of one of the flexible conductive elements 750a-b (e.g., flexible conductive element 750a in FIG. 7). The other flexible conductive element 750a-b (e.g., flexible conductive element 750b in FIG. 7) is insulated from the non-elongatable conductive element 752 by dielectric elements 753 at the cross-over areas. An insulating layer may be added between 752, 753, and/or 750a-b. Without the non-elongatable conductive element 752, the resistance of the flexible conductive elements 750a and 750b is summed like resistors in series, which causes a cumulative voltage drop from the proximal to the distal end of the energy delivery unit 742. This voltage drop can be particularly large for helical flexible conductive elements formed from conductive inks because the resistance increases with expansion of the inflatable member. However, when the non-elongatable conductive element 752 is connected to the distal end of one of the flexible conductive elements 750a-b, the resistance is summed like resistors in parallel. Accordingly, one feature of this arrangement is that the cumulative voltage drop over the length of the energy delivery unit 742 is less than when the supply wires are both connected to the flexible conductive elements at the proximal end of the inflatable member 260.

FIG. 8 is a partially schematic, side cross-sectional view of an energy delivery unit 842 configured in accordance with yet another embodiment of the disclosure. The energy delivery unit 842 is generally similar to the energy delivery unit 642 described above with reference to FIG. 6. For example, the energy unit 842 includes the conductive elements 650a and 650b arranged in an interdigitated or interwoven arrangement around the inflatable member 260. The energy delivery unit 842 differs from the energy delivery unit 642, however, in that the energy delivery unit 842 further includes non-elongatable conductive elements 852a and 852b (e.g., wires, non-compliant base layers with conductive elements) attached to or carried by the inflatable member 260. The non-elongatable conductive elements 852a-b are connected as a backbone to the flexible conductive radial finger elements 850a-b, respectively. Although the interdigitated designs do not experience a cumulative voltage drop to the same extent as the helical design of FIGS. 3A-B, the non-elongatable conductive elements 852a-b can be highly conductive to further reduce the voltage drop over the length of the energy delivery unit 842. In still other embodiments, the non-elongatable conductive elements 752 and 852 can have different dimensions and/or arrangements.

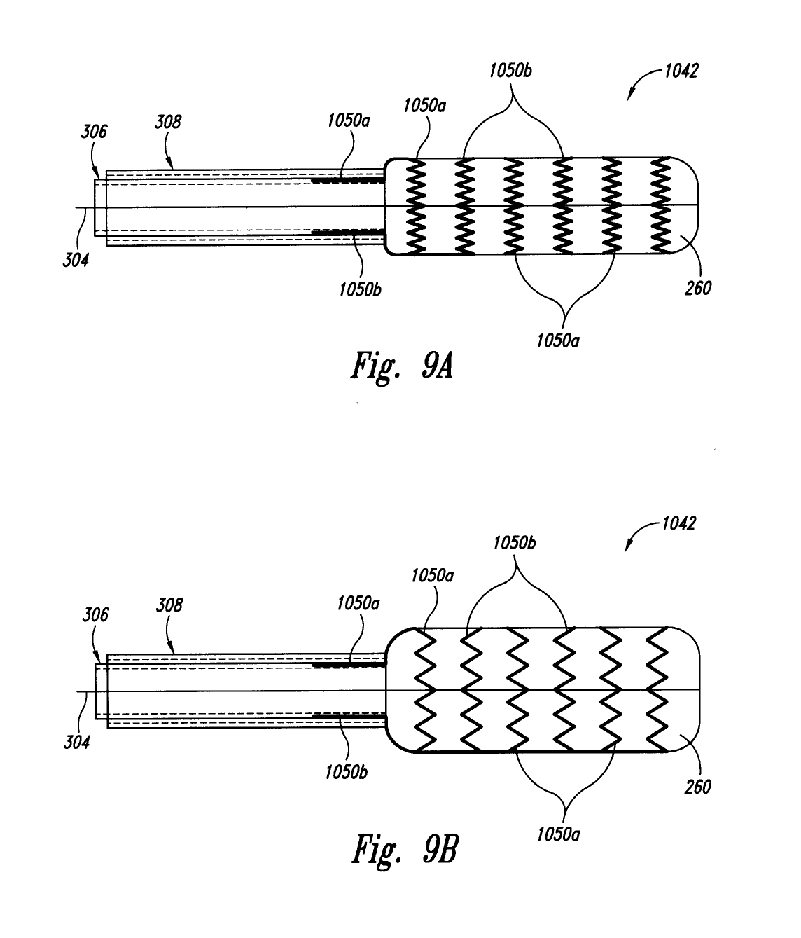

FIGS. 9A-9B are partially schematic, side cross-sectional views illustrating energy delivery units having inelastic, but flexible, expandable conductive elements configured in accordance with other embodiments of the disclosure. More specifically, FIG. 9A illustrates an energy delivery unit 1042 in a retracted or undeployed configuration, and FIG. 9B illustrates the energy delivery unit 1042 in an expanded or deployed configuration. Referring to FIGS. 9A and 9B together, the energy delivery unit 1042 includes expandable conductive elements 1050a and 1050b attached to the inflatable member 260. The conductive elements 1050a and 1050b differ from the conductive elements described above with reference to FIGS. 3A-8 in that the conductive elements 1050a and 1050b are inelastic members (e.g., wires) wrapped around the inflatable member 260 in a desired arrangement that allows the wires to extend/contract longitudinally in length along the path of the conductive elements. In the illustrated embodiment, for example, the conductive elements 1050a and 1050b have a serpentine arrangement to longitudinally expand/contract in accordion-like fashion with the inflatable member 260 between the retracted and expanded configurations.

The energy delivery units described above with reference to FIGS. 2-9B can provide bipolar “electrodes” with controlled electrical properties relative to the length and/or circumference of the applicators. Several embodiments of the systems and methods can accordingly have large electrically conductive contact areas to treat large areas of tissue during a single treatment cycle without extensive edge effects that would otherwise occur with such large monopolar electrical contact areas. As such, several embodiments of the system may reduce the time needed for treating tissue within bronchial airways or other body cavities. This enables a facility to treat more patients and enhances the experience and convenience for the patients. The energy delivery units described herein may also be used in a manner that protects the epithelium of the tissue while controlling the tissue depth and/or penetration of the energy delivery of the device. For example, as described above, the energy delivery units can be cooled to maintain the temperature of the tissue wall of the passageway in a manner that protects the epithelial layer and/or mucosal tissue. Cooling may be applied before, during, and/or after energy delivery.

### E. Conclusion

The headings provided herein are for convenience only and are not intended to limit or interpret the scope or meaning of the claimed technology. Additionally, unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. When the claims use the word “or” in reference to a list of two or more items, that word covers all of the following interpretations of the word: any of the items in the list, all of the items in the list, and any combination of the items in the list.

The foregoing specific embodiments of the disclosure have been described for the purposes of illustration, but various modifications may be made without deviating from the scope of the disclosure. For example, specific features or processes of the various examples described above can be combined to provide further examples. Aspects of the technology may accordingly be modified, if necessary, to employ treatment devices with a plurality of treatment units, thermally conductive devices with various configurations, and concepts of the various patents, applications, and publications incorporated by reference to provide yet further embodiments of the technology. These and other changes, therefore, can be made to the technology in light of the above detailed description. In general, in the following claims, the terms used should not be construed to limit the technology to the specific examples disclosed in the specification and the claims, but should be construed to include all embodiments in accordance with the claims. Accordingly, the technology is not limited by the disclosure, but instead its scope is to be determined entirely by the following claims.

I/We claim:

1. An energy delivery device for treating an airway in a patient, the energy delivery device comprising:
an elongated support configured to be passed through a lumen of a scope;
a compliant inflatable member attached to the elongated support, the inflatable member being configured to move between a collapsed configuration and an expanded configuration; and
a flexible conductive element carried by the inflatable member, wherein the flexible conductive element extends along a path and is configured to expand and contract longitudinally relative to the path as the inflatable member moves between the expanded and collapsed configurations.

2. The energy delivery device of claim 1 wherein the flexible conductive element is elastic.

3. The energy delivery device of claim 1 wherein the flexible conductive element is disposed on an outer surface of the inflatable member, and wherein the flexible conductive element is an elastic conductor composed of a conductive ink having a binder and a conductive filler dispersed in the binder.

4. The energy delivery device of claim 3 wherein the conductive filler comprises electrically conductive flakes, fibers, strands, spheres, rods, cylinders, strips, pellets, or combinations thereof.

5. The energy delivery device of claim 3 wherein the conductive filler comprises silver, copper, carbon, or gold.

6. The energy delivery device of claim 3 wherein the conductive ink is applied to the outer surface of the inflatable member using a pen, a silk screen process, a spraying process, an ink jet printing process and/or a pad print process.

7. The energy delivery device of claim 1 wherein the flexible conductive element has:
a first resistance when the inflatable member is collapsed and the conductive element is in an initial state; and
a second resistance less than ten times the first resistance when the inflatable member is expanded and the conductive element is in a stretched state.

8. The energy delivery device of claim 1 wherein the airway comprises tissue, and wherein the flexible conductive element has a conductivity that is greater than a conductivity of the airway tissue when the inflatable member is in the expanded position and the conductive element is in a stretched state.

9. The energy delivery device of claim 1 wherein the flexible conductive element has:
a first conductivity when the inflatable member is collapsed and the conductive element is in an initial state; and
a second conductivity greater than the first conductivity when the inflatable member is expanded and the conductive element is in a stretched state.

10. The energy delivery device of claim 1 wherein the flexible conductive element is attached to an outer surface of the inflatable member in a helical or ring arrangement.

11. The energy delivery device of claim 1 wherein the flexible conductive element is attached to an outer surface of the inflatable member in an interdigitated arrangement having transverse finger portions and one or more longitudinal finger portions.

12. The energy delivery device of claim 1, further comprising a non-elongatable conductive element attached to the inflatable member and in electrical contact with the flexible conductive element.

13. The energy delivery device of claim 12 wherein the non-elongatable conductive element comprises a wire attached to the inflatable member and coupled to a distal end of the flexible conductive element.

14. The energy delivery device of claim 1 wherein the flexible conductive element is inelastic but expandable.

15. The energy delivery device of claim 14 wherein the flexible conductive element is a wire wrapped in a serpentine path around the inflatable member, and wherein the wire is configured to expand and contract longitudinally relative to the path in an accordion-like fashion as the inflatable member moves between the expanded and collapsed configurations.

16. The energy delivery device of claim 1 wherein the inflatable member is a non-conductive, non-porous balloon.

17. The energy delivery device of claim 1, wherein the flexible conductive element has a length in the range from about 5 mm to about 15 mm.

18. The energy delivery device of claim 1, further comprising a power source that provides RF energy in bipolar fields between adjacent flexible conductive elements.

19. An energy delivery device for treating an airway in a patient, the energy delivery device comprising:
an elongated support configured to be passed through a lumen of a scope;
a compliant inflatable member attached to the elongated support, the compliant inflatable member being configured to move between a collapsed configuration and an expanded configuration through a range of diameters of at least approximately 2 mm to 10 mm; and
an expandable electrode carried by the inflatable member, wherein the expandable electrode comprises a binder and a conductive material dispersed in the binder.

20. The energy delivery device of claim 19 wherein the expandable electrode is configured to longitudinally expand and contract with the compliant inflatable member between the expanded and collapsed configurations.

21. The energy delivery device of claim 19 wherein:
the binder is composed of an elastic polymer material; and
the conductive material is composed of gold, copper, carbon, or silver in the form of flakes, fibers, strands, spheres, rods, cylinders, strips, pellets or combinations thereof.

22. The energy delivery device of claim 19 wherein the conductive material dispersed in the binder includes a plurality of conductive particles at least approximately aligned with each other to minimize the resistance of the expandable electrode when the compliant inflatable member is in the expanded configuration.

23. The energy delivery device of claim 19 wherein the expandable electrode is disposed on an outer surface of the compliant inflatable member in a helical or ring arrangement.

24. The energy delivery device of claim 19 wherein the expandable electrode is disposed on an outer surface of the compliant inflatable member in an interdigitated arrangement having transverse finger portions and one or more longitudinal finger portions.

25. An energy delivery device for treating an airway in a patient, the energy delivery device comprising:
an elongated support configured to be passed through a lumen of a scope;
a compliant inflatable member attached to the elongated support, wherein the inflatable member is configured to move between a collapsed configuration and an expanded configuration; and
a stretchable electrode deposited directly on the inflatable member, wherein the stretchable electrode is configured to increase in length as the inflatable member moves from the collapsed configuration to the expanded configuration.

26. An energy delivery device for use in a body passageway, the energy delivery device comprising:
an elongated body having a proximal portion and a distal portion; and
an energy delivery unit at the distal portion of the elongated body, wherein the energy delivery unit comprises:
a compliant inflatable member moveable between a retracted configuration and an expanded configuration; and
one or more elongatable conductive elements disposed on an outer surface of the inflatable member, wherein the one or more conductive elements are positioned to contact a treatment area within the passageway when the inflatable member is in the expanded configuration.

27. A method for treating asthma, the method comprising:
inserting an energy delivery unit into an airway in a lung of a patient, the energy delivery unit including a compliant inflatable member attached to a distal portion of an elongated support and a flexible conductive element carried by the inflatable member, wherein the flexible conductive element is configured to expand and contract with the inflatable member as the inflatable member moves between a collapsed configuration and an expanded configuration;
expanding the inflatable member such that the flexible conductive element contacts a wall of the airway; and
delivering RF energy to the wall of the airway via the flexible conductive element in a bipolar manner.

28. The method of claim 27 wherein inserting an energy delivery unit into an airway and expanding the inflatable member comprises moving the inflatable member through a range of diameters of at least approximately 2 mm to 10 mm.

29. The method of claim 27 wherein delivering RF energy to the wall of the airway comprises circumferentially and longitudinally heating target tissue at a temperature in a range from about 65° C. to about 70° C. for an activation time period in a range from about 2 seconds to about 3 seconds.

30. The method of claim 29 wherein the target tissue comprises airway smooth muscle.

31. The method of claim 27 wherein delivering RF energy to the wall of the airway reduces airway smooth muscle tissue.

32. The method of claim 27, further comprising cooling a tissue layer adjacent to target tissue of the airway.

33. The method of claim 27 wherein cooling a tissue layer comprises at least partially filling the inflatable member with a liquid or a gas.

34. A method for manufacturing an energy delivery unit for treating an airway in a patient, the method comprising:
expanding a compliant inflatable member to a semi-expanded configuration, wherein the inflatable member is moveable through a range of diameters between a collapsed configuration and a fully expanded configuration; and
applying a conductive ink to an outer surface of the semi-expanded inflatable member, wherein the conductive ink comprises a binder and conductive filler particles dispersed in the binder.

35. The method of claim 34 wherein applying a conductive ink to an outer surface of the semi-expanded inflatable member comprises applying the conductive ink using a pen, a silk screen process, a spraying process, an ink jet printing process and/or a pad print process.

36. The method of claim 34 wherein applying a conductive ink to an outer surface of the semi-expanded inflatable member comprises applying the conductive ink in a helical or ring arrangement around the inflatable member.

37. The method of claim 34 wherein applying a conductive ink to an outer surface of the semi-expanded inflatable member comprises applying the conductive ink in an interdigitated arrangement around the inflatable member.
